(19) United States
(12) Patent Application Publication
(10) Pub. No.: US 2025/0063260 A1
(43) Pub. Date: Feb. 20, 2025

(54) MEASURING INSTRUMENT, MEASURING METHOD, AND COMPUTER-READABLE STORAGE MEDIUM

(71) Applicant: Sharp Kabushiki Kaisha, Osaka (JP)

(72) Inventors: Azusa Nakano, Osaka (JP); Ryota Tomizawa, Osaka (JP); Ken Nakashima, Osaka (JP); Yoshinori Kishibe, Osaka (JP)

(21) Appl. No.: 18/797,800

(22) Filed: Aug. 8, 2024

(30) Foreign Application Priority Data
Aug. 18, 2023 (JP) 2023-133182

(51) Int. Cl. H04N 23/745; H04N 23/73

(52) U.S. Cl. CPC H04N 23/745; H04N 23/73; A61B 2576/00

(57) ABSTRACT

A measuring instrument includes: an image-capturing unit configured to acquire a moving image by performing image capturing on a living organism; a pixel value calculation unit configured to calculate, from each of images constituting the moving image, a representative value of pixel values of pixels in a target region containing an optical image of the living organism; and an image-capturing control unit configured to specify an exposure time and a sensitivity based on an optimal frame rate and the representative value so that the representative value falls within a prescribed range, wherein the image-capturing unit performs the image capturing on the living organism with the optimal frame rate, the exposure time, and the sensitivity.

### CROSS-REFERENCE TO RELATED APPLICATION

The present application claims priority from Japanese Application JP2023-133182, filed on Aug. 18, 2023, the content of which is hereby incorporated by reference into this application.

### BACKGROUND

### 1. Field

The present disclosure relates to measuring instruments, measuring methods, and programs.

### 2. Description of the Related Art

Japanese Unexamined Patent Application Publication No. 2006-094370 discloses a camera-equipped mobile communication terminal device that controls the flickering of the camera by considering frequency information associated with the base station to which the device is connected as frequency information in the location of the device.

### SUMMARY

To acquire biological information by capturing an image of a surface of a living organism, it is desirable to do so with a maximum frame rate that ensures stable operation of the terminal device for the image-capturing and also under brightness suited to acquire biological information. However, the light source may exhibit brightness that periodically changes due to the frequency of the power supply to which the light source is connected, which can in some cases be a cause of flickering being observed that is unwanted periodic noise. It is accordingly preferable to both control the exposure time and adjust the sensitivity so as to reduce the flickering, to capture an image of a surface of a living organism under brightness that is suited to acquire biological information. Hence, the present disclosure, in an aspect thereof, has an object to provide a measuring instrument, a measuring method, and a computer-readable storage medium each of which is capable of capturing an image of a surface of a living organism under brightness that is suited to acquire biological information.

The present disclosure, in an aspect thereof, is directed to a measuring instrument including: an image-capturing unit configured to acquire a moving image by performing image capturing on a living organism; a pixel value calculation unit configured to calculate, from each of images constituting the moving image, a representative value of pixel values of pixels in a target region containing an optical image of the living organism; and an image-capturing control unit configured to specify an exposure time and a sensitivity based on an optimal frame rate and the representative value so that the representative value falls within a prescribed range, wherein the image-capturing unit performs the image capturing on the living organism with the optimal frame rate, the exposure time, and the sensitivity.

The present disclosure, in an aspect thereof, is directed to a measuring method including: a step of acquiring a moving image by performing image capturing on a living organism; a step of calculating, from each of images constituting the moving image, a representative value of pixel values of pixels in a target region containing an optical image of the living organism; and a step of specifying an exposure time and a sensitivity based on an optimal frame rate and the representative value so that the representative value falls within a prescribed range, wherein in the step of acquiring the moving image, the image capturing is performed on the living organism with the optimal frame rate, the exposure time, and the sensitivity.

The present disclosure, in an aspect thereof, is directed to a computer-readable storage medium containing a program configured to cause a computer to perform: a function of acquiring a moving image by performing image capturing on a living organism; a function of calculating, from each of images constituting the moving image, a representative value of pixel values of pixels in a target region containing an optical image of the living organism; a function of specifying an exposure time and a sensitivity based on an optimal frame rate and the representative value so that the representative value falls within a prescribed range; and a function of calculating a pulse wave signal from a temporal change in the representative value, wherein in the function of acquiring the moving image, the image capturing is performed on the living organism with the optimal frame rate, the exposure time, and the sensitivity.

### BRIEF DESCRIPTION OF THE DRAWINGS

FIG. 1 is an illustration of an exemplary usage mode of a measuring instrument.

FIG. 2 is a block diagram of an exemplary structure of a measuring instrument.

FIG. 3 is a diagram showing exemplary flickering-related information.

FIG. 4 is a flow chart representing an exemplary operation of a measuring instrument in accordance with Embodiment 1.

FIG. 5 is a flow chart representing exemplary adjustment of an image-capturing parameter.

FIG. 6 is a flow chart representing exemplary adjustment of the image-capturing parameter, which is a continuation of FIG. 5.

FIG. 7 is a block diagram of an exemplary structure of a measuring instrument in accordance with Embodiment 2.

FIG. 8 is a diagram of an exemplary frame rate table.

FIG. 9 is a flow chart representing an exemplary operation of the measuring instrument in accordance with Embodiment 2.

FIG. 10 is a block diagram of an exemplary structure of an measuring instrument in accordance with Embodiment 3.

FIG. 11 is a flow chart representing an exemplary operation of the measuring instrument in accordance with Embodiment 3.

FIG. 12 is a flow chart representing an exemplary operation of the measuring instrument, which is a continuation of FIG. 11.

FIG. 13 is a block diagram of an exemplary structure of a measuring instrument in accordance with a variation example of Embodiment 3.

### DETAILED DESCRIPTION OF THE DISCLOSURE

### Embodiment 1

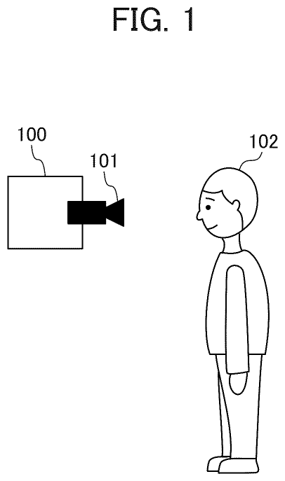

A description is given of Embodiment 1 with reference to FIGS. 1 to 6. Identical and equivalent elements in the drawings are denoted by the same reference numerals, and description thereof is not repeated.

FIG. 1 is an illustration of an exemplary usage mode of a measuring instrument 100. The measuring instrument 100 includes an image-capturing unit 101 shown as an example in FIG. 1.

The measuring instrument 100 calculates a pulse wave signal representing a pulse wave from an image acquired by the image-capturing unit 101. For example, the measuring instrument 100 is, for example, a PC (personal computer), a smartphone, a tablet terminal, or a dedicated pulse wave inferring terminal. In the present specification, a pulse wave is a serial signal representing changes in the volume of a blood vessel that are calculated from a serial signal representing pixel values of pixels in an image related to a single location on a body surface. In the present specification, a pixel value is information representing the brightness of a pixel in an image and, for example, a pixel value of a pixel or a luminance value of a pixel for each color of R (red), G (green), and B (blue).

For instance, the image-capturing unit 101 includes a CCD (charge coupled device) or CMOS (complementary metal oxide semiconductor) image sensor. The image-capturing unit 101 may include an image sensor for camera use that includes RGB (red, green, and blue) filters.

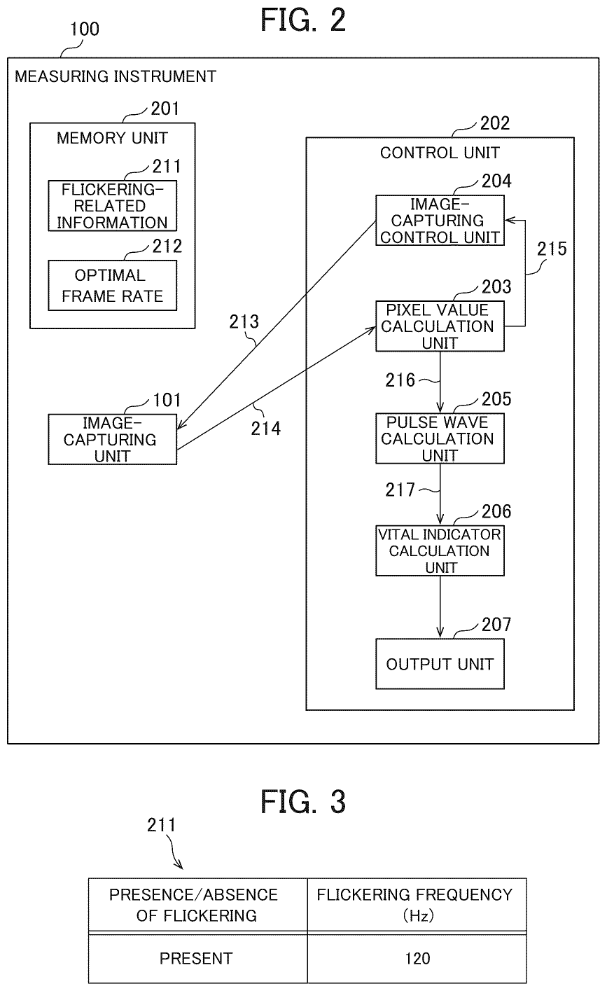

FIG. 2 is a block diagram of an exemplary structure of the measuring instrument 100. The measuring instrument 100 includes the image-capturing unit 101, a memory unit 201, and a control unit 202.

The image-capturing unit 101 captures an image of a living organism 102 to acquire a moving image. Specifically, the image-capturing unit 101 captures an image of the living organism 102 in front of the image-capturing unit 101 to acquire a moving image constituting images including optical images of the body surface of the living organism 102. For example, the optical image of the body surface is, for example, an optical image of the forehead, a cheek, a fingertip, a wrist, or a palm of the living organism 102.

The memory unit 201 is a storage medium capable of recording, for example, various data and programs and may include, for example, a hard disk, a SSD (solid state drive), or a semiconductor memory. The memory unit 201 stores flickering-related information 211 (see FIG. 3) and an optimal frame rate 212.

The control unit 202 performs various processes in accordance with the programs and data stored in the memory unit 201. The control unit 202 includes, for example, a processor such as a CPU (central processing unit). The control unit 202 includes a pixel value calculation unit 203, an image-capturing control unit 204, a pulse wave calculation unit 205, a vital indicator calculation unit 206, and an output unit 207.

The pixel value calculation unit 203 calculates a representative value 215 of pixel values in a target region containing an optical image of the living organism 102 from the images constituting a moving image 214.

The image-capturing control unit 204 specifies an exposure time and a sensitivity on the basis of the optimal frame rate 212 and the representative value 215 so that the representative value 215 can fall within a prescribed range. The image-capturing control unit 204 specifies an exposure time to a value less than or equal to an image-capturing time interval determined from the optimal frame rate. The image-capturing control unit 204 then determines image-capturing parameters 213 including the specified exposure time and the specified sensitivity. The image-capturing control unit 204 acquires the moving image 214 by controlling the image-capturing unit 101 so as to capture an image of the living organism 102 at the optimal frame rate 212 and with the exposure time and sensitivity both included in the image-capturing parameters 213.

The pulse wave calculation unit 205 calculates a pulse wave signal 216 from temporal changes in the representative value 215.

The vital indicator calculation unit 206 calculates a vital indicator of the living organism 102 from the pulse wave signal 216. For example, the vital indicator of the living organism 102 includes, for example, an indicator related to blood pressure and an indicator related to the heart rate.

The output unit 207 outputs information of which the living organism 102 is notified. Specifically, the output unit 207 outputs the vital indicator calculated by the vital indicator calculation unit 206. In addition, when the image-capturing unit 101 is not capable of suitable image-capturing, the output unit 207 outputs a message for notifying that an image of the living organism 102 cannot be suitably captured.

FIG. 3 is a diagram showing an example of the flickering-related information 211. The flickering-related information 211 represents the presence/absence of flickering and a flickering frequency fq. Flickering is periodic changes in the brightness of a light source. When flickering occurs, the pixels contained in the image acquired by the image-capturing unit 101 exhibit periodically changing pixel values.

The flickering-related information 211 shown as an example in FIG. 3 indicates the presence of flickering. Furthermore, the flickering-related information 211 shown as an example in FIG. 3 shows that the flickering frequency fq is 120 Hz. As an example, the flickering frequency fq shown in the flickering-related information 211 has a value in accordance with illumination. For example, in Japan, flickering can occur with a frequency of 120 Hz in the western part of Japan where the power supply is 60 Hz and with a frequency of 100 Hz in the eastern part of Japan where the power supply is 50 Hz. Note that the flickering-related information 211 may not show the flickering frequency fq to indicate the absence of flickering.

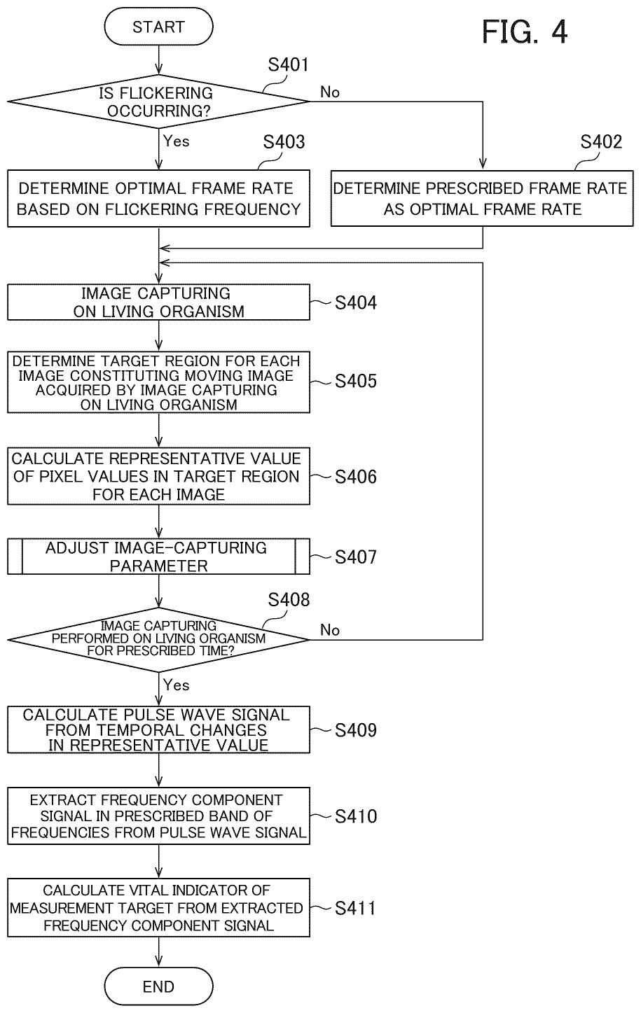

FIG. 4 is a flow chart representing an exemplary operation of the measuring instrument 100 in accordance with the present embodiment. In the present example, when the measuring instrument 100 is activated, the control unit 202 starts step S401 shown as an example in FIG. 4. The flickering-related information 211 is already stored in the memory unit 201 when the control unit 202 starts step S401.

In step S401, the image-capturing control unit 204 determines whether or not flickering is occurring. Specifically, when the flickering-related information 211 indicates the presence of flickering, the image-capturing control unit 204 determines that flickering is occurring. On the other hand, when the flickering-related information 211 indicates the absence of flickering, the image-capturing control unit 204 determines that no flickering is occurring.

If it is determined in step S401 that no flickering is occurring, the image-capturing control unit 204, in step S402, determines a prescribed frame rate as the optimal frame rate 212. For example, the prescribed frame rate is 60 fps (frames per second). The control unit 202 then moves the process to step S404.

On the other hand, if it is determined in step S401 that flickering is occurring, the image-capturing control unit 204, in step S403, determines the optimal frame rate 212 on the basis of the flickering frequency fq. Specifically, if it is determined in step S401 that flickering is occurring, the image-capturing control unit 204 determines the optimal frame rate 212 on the basis of the flickering frequency fq shown by the flickering-related information 211. The control unit 202 then moves the process to step S404.

The image-capturing control unit 204 determines the exposure time included in the image-capturing parameters 213 on the basis of the flickering frequency fq. Specifically, the image-capturing control unit 204 determines the exposure time included in the image-capturing parameters 213 to N/fq seconds. N is a positive integer. As an example, when the flickering frequency fq is 120 Hz, the image-capturing control unit 204 changes the exposure time included in the image-capturing parameters 213 to 1/120 seconds. As another example, when the flickering frequency fq is 100 Hz, the image-capturing control unit 204 changes the exposure time included in the image-capturing parameters 213 to 1/100 seconds.

In step S404, the image-capturing control unit 204 controls the image-capturing unit 101 so that the image-capturing unit 101 can capture an image of the living organism 102 at the optimal frame rate 212 and with the exposure time and sensitivity both included in the image-capturing parameters 213, to acquire the moving image 214. The image-capturing unit 101 then outputs the images constituting the moving image 214 to the pixel value calculation unit 203.

In step S405, the pixel value calculation unit 203 determines a target region for each of the images constituting the moving image 214. A target region is a part of the body surface area in the images constituting the moving image 214 and contains a plurality of pixels. As an example, when the image constituting the moving image 214 contains a facial area of the living organism 102, the target region contains a cheek segment, a forehead segment, or a glabella segment. Either a single target region or a plurality of target regions may be involved. The target region may be surrounded either by straight lines like a polygonal or by curved lines. As a further alternative, the target region may be a closed region surrounded by straight and curved lines.

In step S406, the pixel value calculation unit 203 calculates the representative value 215 of the pixel values in the target region determined in step S405 for the images constituting the moving image 214. The representative value 215 is, for example, the average value, median value, or mode value of the pixel values of pixels in the target region. Alternatively, when the image-capturing unit 101 includes image sensors for camera use that include RGB filters, the pixel value calculation unit 203 may calculate a representative R, G, and B values of the pixel values.

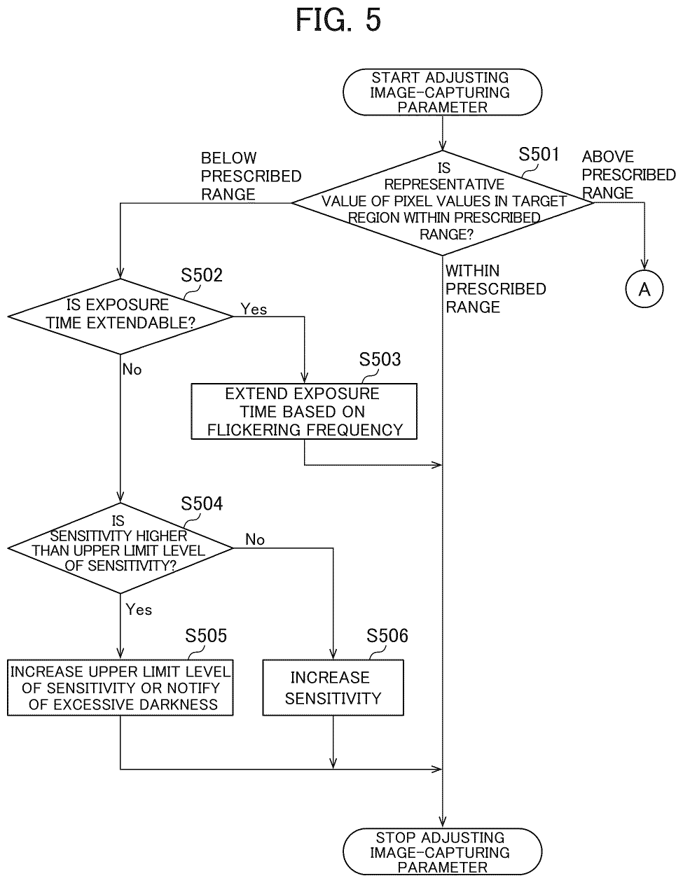

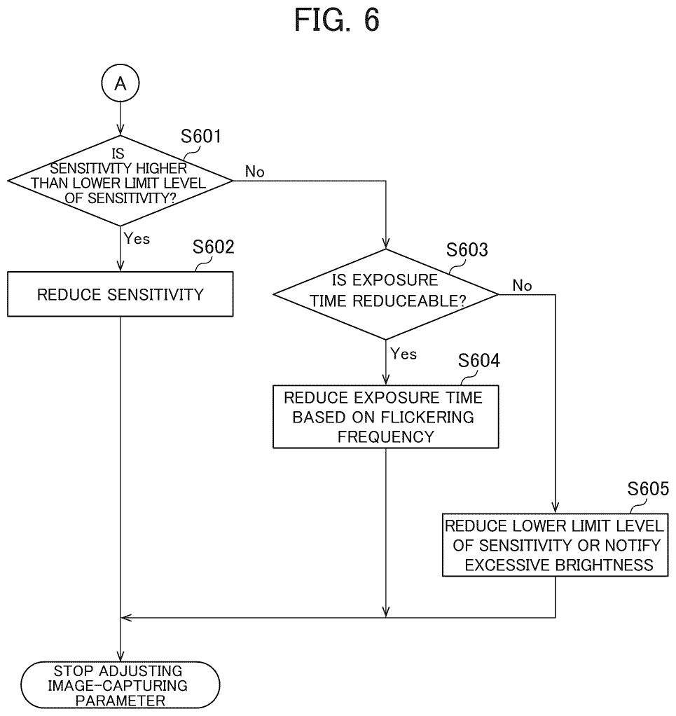

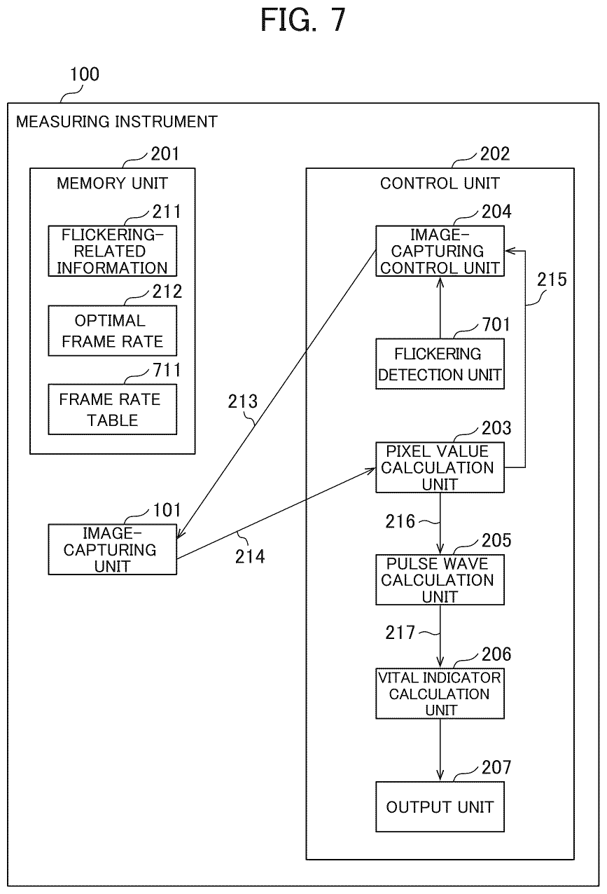

In step S407, the image-capturing control unit 204 adjusts the image-capturing parameters 213 on the basis of the optimal frame rate 212 and the representative value 215. Specifically, the image-capturing control unit 204 adjusts the exposure time and sensitivity both included in the image-capturing parameters 213 on the basis of the optimal frame rate 212 and the representative value 215 so that the representative value 215 can fall within a prescribed range. The adjustment of the image-capturing parameters 213 will be detailed with reference to FIGS. 5 to 6.

In step S408, the image-capturing control unit 204 determines whether or not the image-capturing unit 101 has been capturing an image of the living organism 102 for a prescribed time. If the image-capturing unit 101 has not been capturing an image of the living organism 102 for the prescribed time in step S409, the control unit 202 returns the process to step S404. Hence, the image-capturing control unit 204 repeats the adjustment of the image-capturing parameters 213 in the prescribed time in step S407 and controls the image-capturing unit 101 to acquire the moving image 214.

On the other hand, if the image-capturing unit 101 has been capturing an image of the living organism 102 for the prescribed time in step S408, the pulse wave calculation unit 205, in step S409, calculates the pulse wave signal 216 from the temporal changes in the representative value 215. Specifically, the pulse wave calculation unit 205 calculates the pulse wave signal 216 from the temporal changes in the representative value 215 that correspond to a single location on the body surface area. For example, the pulse wave calculation unit 205 processes a signal that represents the temporal changes in the representative value 215 by multivariable analysis, for example, by principle component analysis or by independent component analysis and calculates results of the process as the pulse wave signal 216. The temporal changes in the representative value contain information on changes in the volume of the blood vessel.

In step S410, the vital indicator calculation unit 206 calculates a vital indicator to be measured from the pulse wave signal 216. As an example, when the type of the vital indicator to be measured is blood pressure, the vital indicator calculation unit 206 calculates a maximum blood pressure on the basis of a rising angle of the pulse wave signal 216. As another example, when the type of the vital indicator to be measured is heart rate, the vital indicator calculation unit 206 calculates a heart rate on the basis of the number of peaks on the pulse wave signal 216.

A detailed description is given next of the adjustment of the image-capturing parameters 213 with reference to FIGS. 5 to 6. FIGS. 5 to 6 are flow charts representing an exemplary adjustment of the image-capturing parameters 213.

In step S501, the image-capturing control unit 204 determines whether or not the representative value 215 calculated in step S406 shown as an example in FIG. 4 is in a prescribed range. For example, the image-capturing control unit 204 determines whether or not the representative values 215 calculated in step S406 shown as an example in FIG. 4 for the respective images in the plurality of frames constituting the moving image 214 are in a prescribed range.

Alternatively, the image-capturing control unit 204 may determine whether or not an average value of the representative values 215 calculated in step S406 shown as an example in FIG. 4 for the respective images in the plurality of frames constituting the moving image 214 is in a prescribed range. Alternatively, the image-capturing control unit 204 may determine whether or not the representative value 215 for the image in one of the frames selected from the representative values 215 calculated in step S406 for the respective images in the plurality of frames constituting the moving image 214 is in a prescribed range.

If the representative value 215 is in a prescribed range in step S401, the control unit 202 moves the process to step S408 shown as an example in FIG. 4 without, adjusting the image-capturing parameters. Meanwhile, if the representative value 215 is greater than the prescribed range in step S501, the control unit 202 moves the process to step S601 shown as an example in FIG. 6.

If the representative value 215 is below the prescribed range in step S501, the image-capturing control unit 204, in step S502, determines whether or not it is possible to extend the exposure time included in the image-capturing parameters 213. If the exposure time included in the image-capturing parameters 213 is shorter than the image-capturing time interval determined from the optimal frame rate 212, the image-capturing control unit 204 determines that it is possible to extend the exposure time. As an example, when the optimal frame rate 212 is 60 fps and the exposure time is shorter than 1/60 seconds, the image-capturing control unit 204 determines that it is possible to extend the exposure time. On the other hand, if the exposure time included in the image-capturing parameters 213 is greater than or equal to the image-capturing time interval determined from the optimal frame rate 212, the image-capturing control unit 204 determines that it is not possible to extend the exposure time.

If the image-capturing control unit 204 determines in step S502 that it is possible to extend the exposure time, the image-capturing control unit 204, in step S503, extends the exposure time on the basis of the flickering frequency fq. In other words, through steps S501 to S503, if the representative value 215 is below the prescribed range and the exposure time included in the image-capturing parameters 213 is shorter than the image-capturing time interval determined from the optimal frame rate 212, the image-capturing control unit 204 adjusts the exposure time included in the image-capturing parameters 213.

Specifically, the adjustment by the image-capturing control unit 204 of the exposure time included in the image-capturing parameters 213 is to select any of a plurality of candidate exposure times calculated from the optimal frame rate 212 to specify the exposure time included in the image-capturing parameters 213. For example, the image-capturing control unit 204 extends the exposure time included in the image-capturing parameters 213 by changing the exposure time included in the image-capturing parameters 213 from N/fq to (N+1)/fq, to adjust the exposure time included in the image-capturing parameters 213. N is a positive integer. As an example, when the flickering frequency fq is 120 Hz, and the exposure time included in the image-capturing parameters 213 is 1/120 seconds, the image-capturing control unit 204 changes the exposure time included in the image-capturing parameters 213 to 2/120. As another example, when the flickering frequency fq is 100 Hz, and the exposure time included in the image-capturing parameters 213 is 1/100 seconds, the image-capturing control unit 204 changes the exposure time included in the image-capturing parameters 213 to 1/50 (=2/100) seconds. The image-capturing control unit 204 hence specifies the exposure time to a value less than or equal to the image-capturing time interval determined from the optimal frame rate 212.

Then, after the image-capturing control unit 204 extends the exposure time on the basis of the flickering frequency fq, the control unit 202 terminates the adjustment of the image-capturing parameters 213 and moves the process to step S408 shown as an example in FIG. 4.

If the image-capturing control unit 204 determines in step S502 that it is not possible to extend the exposure time, the image-capturing control unit 204, in step S504, determines whether or not the sensitivity included in the image-capturing parameters 213 is lower than or equal to an upper limit level of sensitivity. Assume that the upper limit level of sensitivity is specified in accordance with the measuring instrument 100 and stored in the memory unit 201.

If the sensitivity included in the image-capturing parameters 213 is higher than the upper limit level in step S504, the image-capturing control unit 204, in step S505, causes the memory unit 201 to store a new upper limit level of sensitivity that is higher than the upper limit level of sensitivity stored in the memory unit 201. Alternatively, the image-capturing control unit 204, in step S505, causes the output unit 207 to output a message for notifying that the image-capturing environment is too dark for suitable image-capturing. The output unit 207 may cause a display device (not shown) connected to the measuring instrument 100 to display a text message for notifying that the image-capturing environment is too dark for suitable image-capturing. Alternatively, the output unit 207 may cause a speaker (not shown) connected to the measuring instrument 100 to output an audio message for notifying that the image-capturing environment is too dark for suitable image-capturing. The control unit 202 then terminates the adjustment of the image-capturing parameters 213 and moves the process to step S408 shown as an example in FIG. 4.

On the other hand, if the sensitivity included in the image-capturing parameters 213 is lower than or equal to the upper limit level in step S504, the image-capturing control unit 204, in step S506, adjusts the sensitivity included in the image-capturing parameters 213 by specifying a new sensitivity that exceeds the sensitivity included in the image-capturing parameters 213 as the sensitivity included in the image-capturing parameters 213. In other words, the adjustment by the image-capturing control unit 204 of the sensitivity included in the image-capturing parameters 213 in step S506 is to specify a new sensitivity that exceeds the sensitivity included in the image-capturing parameters 213 if the representative value 215 is below the prescribed range, the exposure time included in the image-capturing parameters 213 is greater than or equal to the image-capturing time interval, and the sensitivity included in the image-capturing parameters 213 is lower than the upper limit level of sensitivity. Hence, through steps S501 to S502, S504, and S506, when the representative value 215 is below the prescribed range, and the exposure time included in the image-capturing parameters 213 is greater than or equal to the image-capturing time interval determined from the optimal frame rate 212, the image-capturing control unit 204 adjusts the sensitivity included in the image-capturing parameters 213. The control unit 202 then terminates the adjustment of the image-capturing parameters 213 and moves the process to step S408 shown as an example in FIG. 4.

A description is given next of a process performed when the representative value 215 is above the prescribed range with reference to FIG. 6. FIG. 6 is a flow chart representing exemplary adjustment of the image-capturing parameters 213, which is a continuation of FIG. 5.

If the representative value 215 is above the prescribed range in step S501 shown as an example in FIG. 5, it is determined in step S601 whether or not the sensitivity included in the image-capturing parameters 213 is higher than a lower limit level of sensitivity. Assume that the lower limit level of sensitivity is specified in accordance with the measuring instrument 100 and stored in the memory unit 201.

If the sensitivity included in the image-capturing parameters 213 is higher than the lower limit level of sensitivity in step S601, the image-capturing control unit 204, in step S602, adjusts the sensitivity included in the image-capturing parameters 213 by determining a new sensitivity that is lower than the sensitivity included in the image-capturing parameters 213 as the sensitivity included in the image-capturing parameters 213. In other words, the adjustment by the image-capturing control unit 204 of the sensitivity included in the image-capturing parameters 213 is to specify a new sensitivity that is lower than sensitivity if the representative value 215 is above the prescribed range, and the sensitivity included in the image-capturing parameters 213 is higher than the lower limit level of sensitivity. Hence, through steps S501 and S601 to S602 shown as an example in FIG. 5, when the representative value 215 is above the prescribed range, and the sensitivity included in the image-capturing parameters 213 is higher than the lower limit level of sensitivity, the image-capturing control unit 204 adjusts the sensitivity included in the image-capturing parameters. The control unit 202 then terminates the adjustment of the image-capturing parameters 213 and moves the process to step S408 shown as an example in FIG. 4.

On the other hand, if the sensitivity included in the image-capturing parameters 213 is lower than or equal to the lower limit level of sensitivity in step S601, the image-capturing control unit 204, in step S603, determines whether or not it is possible to reduce the exposure time included in the image-capturing parameters 213.

If the exposure time included in the image-capturing parameters 213 is longer than the lower limit level of exposure time that can be specified on the measuring instrument 100, the image-capturing control unit 204 determines that it is possible to reduce the exposure time. Assume that the lower limit level of exposure time is specified in accordance with the measuring instrument 100 and stored in the memory unit 201. For example, when the lower limit level of exposure time is 80 milliseconds, and the exposure time included in the image-capturing parameters 213 is longer than 80 milliseconds, the image-capturing control unit 204 determines that it is possible to reduce the exposure time. On the other hand, if the exposure time included in the image-capturing parameters 213 is shorter than the lower limit level of exposure time, the image-capturing control unit 204 determines that it is not possible to reduce the exposure time.

If the image-capturing control unit 204 determines in step S603 that it is possible to reduce the exposure time, the image-capturing control unit 204, in step S604, reduces the exposure time on the basis of the flickering frequency fq. In other words, through steps S501, S601, and S603 to S604, the image-capturing control unit 204 adjusts the exposure time included in the image-capturing parameters 213 if the representative value 215 is above the prescribed range, and the sensitivity included in the image-capturing parameters 213 is lower than or equal to the lower limit level of sensitivity.

Specifically, the adjustment by the image-capturing control unit 204 of the exposure time included in the image-capturing parameters 213 is to select any of a plurality of candidate exposure times calculated from the optimal frame rate 212 to specify the exposure time included in the image-capturing parameters 213. For example, the image-capturing control unit 204 reduces the exposure time included in the image-capturing parameters 213 by changing the exposure time included in the image-capturing parameters 213 from N/fq to (N−1)/fq. N is a positive integer. As an example, when the flickering frequency fq is 120 Hz, and the exposure time included in the image-capturing parameters 213 is 1/60 (=2/120) seconds, the image-capturing control unit 204 changes the exposure time included in the image-capturing parameters 213 to 1/120. As another example, when the flickering frequency fq is 100 Hz, and the exposure time included in the image-capturing parameters 213 is 1/50 (=2/100) seconds, the image-capturing control unit 204 changes the exposure time included in the image-capturing parameters 213 to 1/100 (=1/100) seconds. The image-capturing control unit 204 hence specifies the exposure time to a value less than or equal to the image-capturing time interval determined from the optimal frame rate 212.

After the image-capturing control unit 204 reduces the exposure time on the basis of the flickering frequency fq, the control unit 202 terminates the adjustment of the image-capturing parameters 213 and moves the process to step S408 shown as an example in FIG. 4.

On the other hand, if the image-capturing control unit 204 determines in step S603 that it is not possible to reduce the exposure time, the image-capturing control unit 204, in step S604, causes the memory unit 201 to store a new lower limit level of sensitivity that is lower than the lower limit level of sensitivity stored in the memory unit 201. Alternatively, the image-capturing control unit 204, in step S604, causes the output unit 207 to output a message for alarming to the living organism 102 that the image-capturing environment is too bright for suitable image-capturing. The output unit 207 may cause a display device (not shown) connected to the measuring instrument 100 to display a text message for alarming to the living organism 102. Alternatively, the output unit 207 may cause a speaker (not shown) connected to the measuring instrument 100 to output an audio message for alarming to the living organism 102. The control unit 202 then terminates the adjustment of the image-capturing parameters 213 and moves the process to step S408 shown as an example in FIG. 4.

The measuring instrument 100 in accordance with the present embodiment is hence capable of adjusting the sensitivity and controlling the exposure time so as to reduce flickering, to capture an image of the surface of the living organism under the brightness that is suited to acquire biological information.

### Embodiment 2

A description is given of Embodiment 2 with reference to FIGS. 7 to 9. Identical and equivalent elements in the drawings are denoted by the same reference numerals, and description thereof is not repeated. The members and processes of the present embodiment that have practically the same arrangement and function as the members and processes of other embodiments are denoted by the same reference numerals, and description thereof is omitted. The description will focus on differences from the other embodiments.

FIG. 7 is a block diagram of an exemplary structure of the measuring instrument 100 in accordance with the present embodiment. The measuring instrument 100 shown as an example in FIG. 7 differs from the measuring instrument 100 shown as an example in FIG. 2 in that the measuring instrument 100 shown as an example in FIG. 7 includes a flickering detection unit 701 and that the memory unit 201 stores a frame rate table 711.

The flickering detection unit 701 detects at least one species selected from the group consisting of the presence/absence of flickering and a flickering frequency.

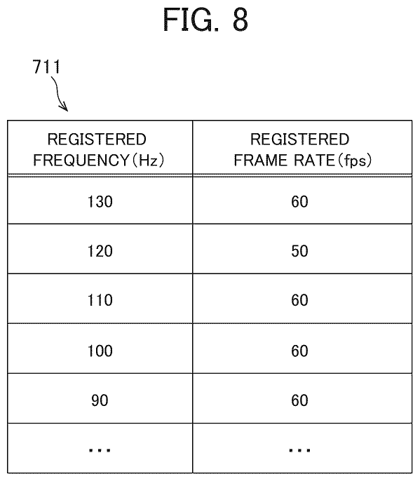

FIG. 8 is a diagram of an example of the frame rate table 711. In the frame rate table 711, a registered frame rate is associated with a registered frequency. Specifically, in the frame rate table 711, a frame rate for which the absolute value of the difference between a registered frequency and an integral multiple of a registered frame rate is greater than or equal to a threshold value is registered as the registered frame rate.

As an example, when the registered frequency is 120 Hz, the registered frame rate is 50 fps. As another example, when the registered frequency is 100 Hz, the registered frame rate is 60 fps. Note that the registered frequencies and the registered frame rates shown as an example in FIG. 8 are mere examples and that there is no intention at all to limit the registered frequency and the registered frame rate to the numerical values shown as an example in FIG. 8.

For instance, registered frequencies and registered frame rates may be registered in the frame rate table 711 in an associated manner, for example, for each manufacturer and model of the measuring instrument 100. Alternatively, registered frequencies and registered frame rates may be registered in the frame rate table 711 in an associated manner by the user entering the registered frequencies and the registered frame rates through an operation unit (not shown) provided in the measuring instrument 100.

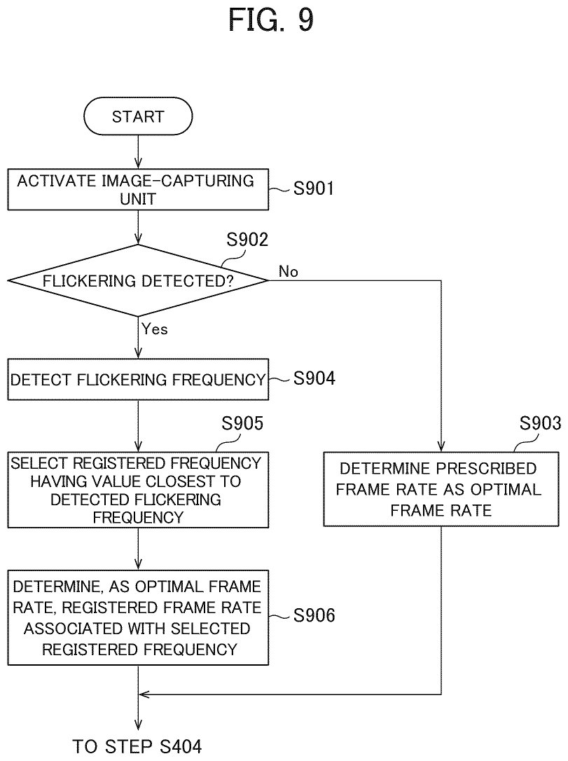

FIG. 9 is a flow chart representing an exemplary operation of the measuring instrument 100 in accordance with the present embodiment. In the present example, the control unit 202 starts step S901 shown as an example in FIG. 9 when the measuring instrument 100 is activated. The frame rate table 711 is already stored in the memory unit 201 when the control unit 202 starts step S901 shown as an example in FIG. 9.

In step S901, the image-capturing control unit 204 activates the image-capturing unit 101. Upon the activation, the image-capturing unit 101 starts the process of performing image-capturing across an image-capturing range to acquire an image. The image acquired in step S901 is for detecting the presence/absence of flickering. When the image-capturing unit 101 captures an image to detect the presence/absence of flickering, the living organism 102 may not be present in the image-capturing range.

In step S902, the flickering detection unit 701 determines whether or not flickering has been detected. For example, the flickering detection unit 701 calculates a luminance value from the pixel values of the pixels in a prescribed region included in the image captured to detect the presence/absence of flickering. A prescribed region is a region that is different from the body surface. In this case, the flickering detection unit 701 detects the timings of peak temporal changes in the luminance value from a time-series signal representing temporal changes in the calculated luminance value. If the timings of peak temporal changes in the luminance value are cyclic, the flickering detection unit 701 determines that flickering has been detected. For example, if the timings of peak temporal changes in the luminance value have a cycle attributable to predetermined flickering, the flickering detection unit 701 determines that flickering has been detected. On the other hand, if the timings of peak temporal changes in the luminance value is not cyclic, the flickering detection unit 701 determines that no flickering has been detected. Note that the flickering detection unit 701 may determine that flickering has been detected by capturing an image of the image-capturing range at a plurality of frame rates and if the timings of peak temporal changes in the luminance value have different cycles from one frame rate to the other.

If no flickering is detected in step S902, the image-capturing control unit 204, in step S903, determines a prescribed frame rate as the optimal frame rate 212. Step S903 is substantially the same as step S402 shown as an example in FIG. 4, and detailed description thereof is therefore omitted. The control unit 202 then moves the process to step S404 shown as an example in FIG. 4.

On the other hand, if flickering is detected in step S902, the flickering detection unit 701, in step S904, detects the flickering frequency fq. For example, upon detection of timings of peak temporal changes in the luminance value, the flickering detection unit 701 detects the flickering frequency fq from the time interval between detected timings. Alternatively, the flickering detection unit 701 may calculate a frequency spectrum related to the time-series signal representing temporal changes in the luminance value. The flickering detection unit 701 may then detect, as the flickering frequency fq, a peak frequency in a prescribed band of frequencies in the calculated frequency spectrum.

In step S905, the image-capturing control unit 204 selects the registered frequency that has the closest value to the flickering frequency fq detected in step S904 in the frame rate table 711.

In step S906, the image-capturing control unit 204 determines, as the optimal frame rate 212, the registered frame rate associated with the selected registered frequency in the frame rate table 711. The control unit 202 then moves the process to step S404 shown as an example in FIG. 4.

The measuring instrument 100 in accordance with the present embodiment hence detects the presence/absence of flickering and captures an image of the living organism 102 at a pre-registered frame rate in accordance with the flickering frequency fq. The measuring instrument 100 in accordance with the present embodiment is hence capable of restraining the influence of noise caused by flickering and also capturing an image of the living organism 102 at the highest frame rate possible.

### Embodiment 3

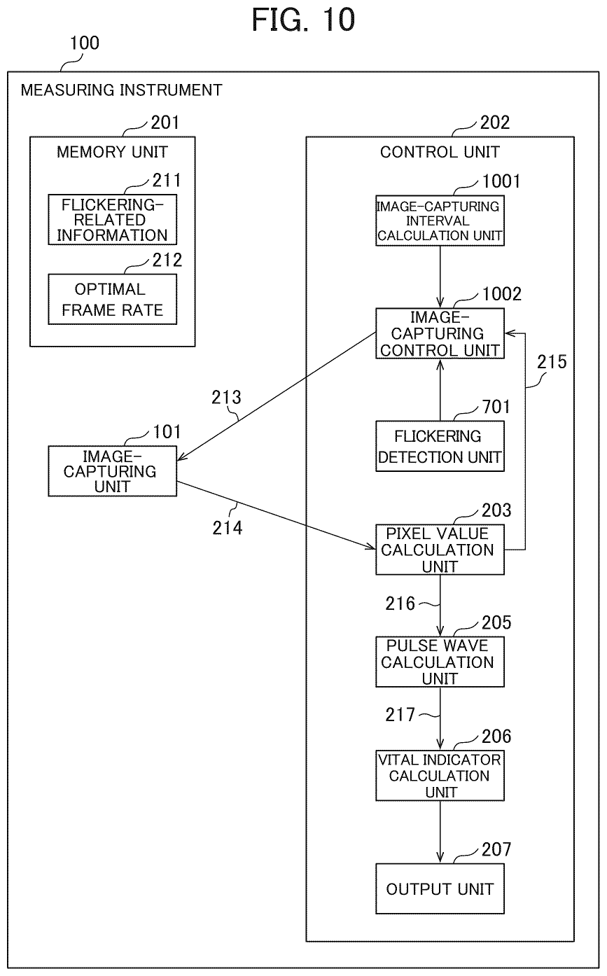

A description is given of Embodiment 3 with reference to FIGS. 10 to 12. Identical and equivalent elements in the drawings are denoted by the same reference numerals, and description thereof is not repeated. The members and processes of the present embodiment that have practically the same arrangement and function as the members and processes of other embodiments are denoted by the same reference numerals, and description thereof is omitted. The description will focus on differences from the other embodiments.

FIG. 10 is a block diagram of an exemplary structure of the measuring instrument 100 in accordance with the present embodiment. The measuring instrument 100 shown as an example in FIG. 10 differs from the measuring instrument 100 shown as an example in FIG. 7 in that the measuring instrument 100 shown as an example in FIG. 10 includes an image-capturing interval calculation unit 1001 and includes an image-capturing control unit 1002 in place of the image-capturing control unit 204.

The image-capturing interval calculation unit 1001 calculates an actual frame rate fr from a difference between the times at which the images constituting the moving image 214 are acquired.

The image-capturing control unit 1002 controls the settings of the image-capturing unit 101 in accordance with a difference between the actual frame rate fr and the optimal frame rate 212. Specifically, the image-capturing control unit 1002 controls the settings of the image-capturing unit 101 so that the difference between the actual frame rate fr and the optimal frame rate 212 can fall in a prescribed range.

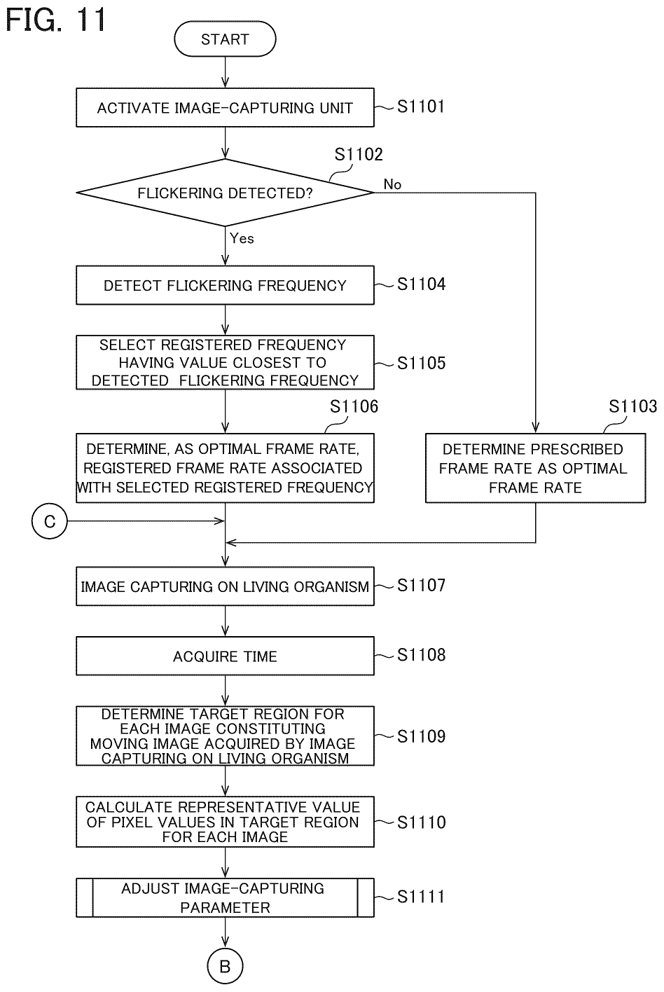

FIG. 11 is a flow chart representing an exemplary operation of the measuring instrument 100 in accordance with the present embodiment. Steps S1101 to S1106 shown as an example in FIG. 11 are substantially the same as steps S901 to S906 shown as an example in FIG. 9, and detailed description thereof is therefore omitted.

In step S1107, the image-capturing control unit 1002 acquires an image by controlling the image-capturing unit 101 so as to capture an image of the living organism 102 with the exposure time and sensitivity included in the image-capturing parameters 213. The image-capturing unit 101 then outputs the images constituting the acquired moving image to the pixel value calculation unit 203.

In step S1108, the image-capturing control unit 1002 acquires the times at which the images constituting the moving image were acquired in step S1107. For example, when the image-capturing control unit 1002 has acquired the times at which the images were acquired, the image-capturing control unit 1002 causes the memory unit 201 to store time information by which the times at which these images were acquired are associated with the frame numbers assigned to the images.

In step S1109, the pixel value calculation unit 203 determines a target region for each of the images constituting the acquired moving image. In step S1110, the pixel value calculation unit 203 calculates the representative value 215 of the pixel values in the target region for each image constituting the acquired moving image. Steps S1109 to S1110 are substantially the same as steps S405 to S406 shown as an example in FIG. 4, and detailed description thereof is therefore omitted.

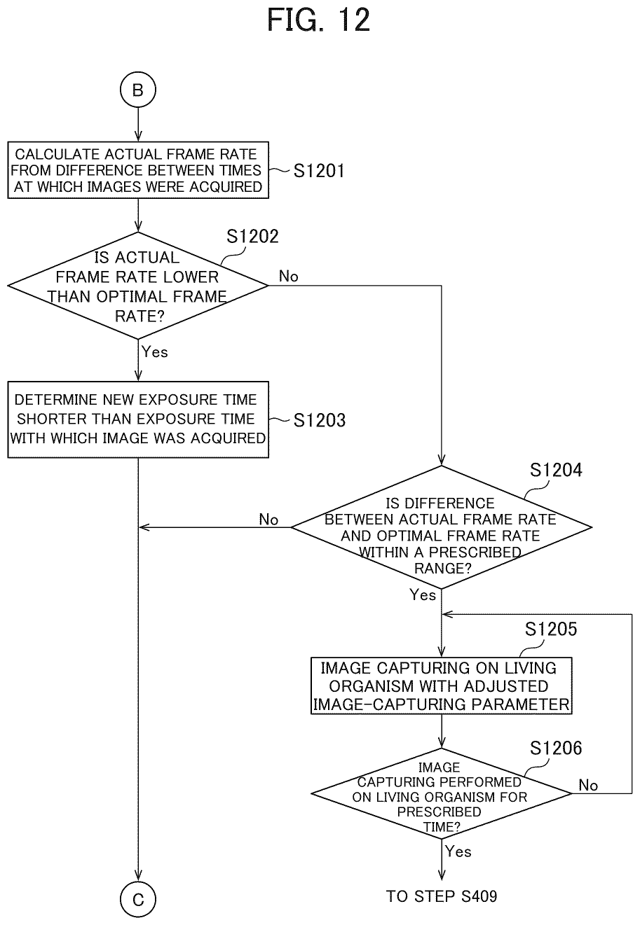

In step S1111, the image-capturing control unit 204 adjusts the image-capturing parameters 213 on the basis of the optimal frame rate 212 determined in step S1103 or S1106 and a representative value 215 calculated in step S1110. Details of the adjustment of the image-capturing parameters 213 are as shown as an example in FIGS. 5 to 6, detailed description thereof is therefore omitted. The control unit 202 then, moves the process to step S1201 shown as an example in FIG. 12.

FIG. 12 is a flow chart representing an exemplary operation of the measuring instrument 100, which is a continuation of FIG. 11.

In step S1201, the image-capturing interval calculation unit 1001 calculates the actual frame rate fr from the time difference between the times acquired in step S1108 shown as an example in FIG. 11 for images in a prescribed number of frames. For example, the image-capturing interval calculation unit 1001 calculates the difference between times at which images in a prescribed number of frames were acquired, by acquiring time information from the memory unit 201.

For instance, when the prescribed number of frames is two, the image-capturing interval calculation unit 1001 calculates, as the time difference, the absolute value of the difference between time t₁ and time t₀ at which the images in two successive frames were acquired. Time to is the time at which an image in a frame was acquired at an earlier time of the two successive frames. Time t₁ is the time at which an image in a frame was acquired at a time later than time to of the two successive frames. The image-capturing interval calculation unit 1001 then calculates the reciprocal of the calculated time difference as the actual frame rate fr. In other words, the image-capturing interval calculation unit 1001 calculates the actual frame rate fr by using a calculation formula: fr=1/(t₁−t₀).

Alternatively, for example, when the prescribed number of frames is three or greater, the actual frame rate fr may be calculated from a time difference in acquiring images in two successive frames of the three or more frames.

Assume, for instance, that the time at which an image in each frame was acquired is time t₁ when the prescribed number of frames is N. N is an integer greater than or equal to 3, and i is an integer greater than or equal to 0 and less than or equal to N−1. In such a case, the frame rates for two successive frames of the N frames are respectively 1/(tₖ−tₖ−1), where k is an integer more than or equal to 1 and less than or equal to N−1. The image-capturing interval calculation unit 1001 calculates, as the actual frame rate fr, a representative value of the frame rates related to the two successive frames of the N frames. For example, the image-capturing interval calculation unit 1001 calculates the actual frame rate fr by using a calculation formula: fr=(Σ^{N-1}k=1 (1/(tₖ-tₖ−1)))/(N−1). For example, when N=4, the image-capturing interval calculation unit 1001 calculates the actual frame rate fr by using a calculation formula: fr=(1/(t₃−t₂)+1/(t₂−t₁)+1/(t₁−t₀))/3.

Alternatively, the image-capturing interval calculation unit 1001 may calculate the actual frame rate fr by using a calculation formula: fr=(N−1)/(tN−1−t₀). For example, when the prescribed number of frames is 30, the image-capturing interval calculation unit 1001 calculates the actual frame rate fr by using a calculation formula: fr=29/(t₂₉−t₀).

In step S1202, the image-capturing control unit 1002 determines whether or not the actual frame rate fr is lower than the optimal frame rate 212. If the actual frame rate fr is lower than the optimal frame rate 212 in step S1202, the image-capturing control unit 1002, in step S1203, determines a new exposure time that is shorter than the exposure time with which the images constituting the moving image acquired in step S1107 shown as an example in FIG. 11 were acquired. By the image-capturing control unit 1002 rendering a new exposure time ET2 shorter than the exposure time with which the images constituting the acquired moving image were acquired, an actual frame rate fr2 for subsequent processes is rendered higher than an actual frame rate fr1 at which images were acquired in step S1107. The control unit 202 then returns the process to step S1107 shown as an example in FIG. 11.

On the other hand, if the actual frame rate fr is greater than or equal to the optimal frame rate 212 in step S1202, the image-capturing control unit 1002, in step S1204, determines whether or not the difference between the actual frame rate fr and the optimal frame rate 212 is within a prescribed range.

If the difference between the actual frame rate fr and the optimal frame rate 212 is not within a prescribed range in step S1204, the control unit 202 returns the process to step S1107 shown as an example in FIG. 11. On the other hand, if the difference between the actual frame rate fr and the optimal frame rate 212 is within the prescribed range in step S1204, the image-capturing control unit 1002, in step S1205, controls the image-capturing unit 101 to capture an image of the living organism 102 with an optimal frame rate determined in step S1103 or step S1106 shown as an example in FIG. 11, the exposure time included in the image-capturing parameters 213 and adjusted in step S1111 shown as an example in FIG. 11, and the sensitivity included in the image-capturing parameters 213 and adjusted in step S1111, to acquire the moving image 214. The image-capturing unit 101 outputs the images constituting the acquired moving image 214 to the pixel value calculation unit 203.

In step S1206, the image-capturing control unit 204 determines whether or not the image-capturing unit 101 captured an image of the living organism 102 for a prescribed time. If the image-capturing unit 101 did not capture an image of the living organism 102 for a prescribed time in step S1206, the control unit 202 returns the process to step S1205. On the other hand, if the image-capturing unit 101 captured an image of the living organism 102 for a prescribed time in step S1206, the control unit 202 moves the process to step S409 shown as an example in FIG. 4.

The measuring instrument 100 in accordance with the present embodiment hence adjusts the image-capturing parameters 213 in accordance with the actual frame rate fr in such a manner that the image-capturing parameters 213 can approach the optimal frame rate 212. The measuring instrument 100 in accordance with the present embodiment can hence adjust sensitivity and control the exposure time so as to reduce flickering by taking a processing time for the image-capturing unit 101 and a processing time in the pixel value calculation unit 203, to capture an image of the surface of a living organism under brightness that is suited to acquire biological information even if the actual frame rate fr has decreased to below the optimal frame rate 212.

### Variation Example 1 of Embodiment 3

As Variation Example 1 of the measuring instrument 100 in accordance with the present embodiment, the image-capturing control unit 1002 may determine the optimal frame rate 212 in accordance with the flickering frequency fq by using a prescribed calculation formula. For example, the image-capturing control unit 1002 may determine, as the optimal frame rate 212, a value obtained by dividing the flickering frequency fq by integer N. For example, if the flickering frequency fq is 120 Hz, the image-capturing control unit 1002 may determine 60 fps (=120/2) as the optimal frame rate 212. Alternatively, for example, if the flickering frequency fq is 100 Hz, the image-capturing control unit 1002 may determine 50 fps (=100/2) as the optimal frame rate 212.

### Variation Example 2 of Embodiment 3

As Variation Example 2 of the measuring instrument 100 in accordance with the present embodiment, if the upper limit level of the frame rate is allowed to be specified, the image-capturing control unit 1002 may determine the upper limit level of the frame rate as the optimal frame rate 212 determined in step S1103 or S1106. The image-capturing control unit 1002 can hence prevent the actual frame rate fr from exceeding the optimal frame rate 212. As a result, the image-capturing control unit 1002 can more readily render the difference between the actual frame rate fr and the optimal frame rate 212 within the prescribed range.

### Variation Example 3 of Embodiment 3

As Variation Example 3 of the measuring instrument 100 in accordance with the present embodiment, the image-capturing control unit 1002 may adjust the exposure time at intervals that are greater than or equal to a prescribed time. If the difference between the actual frame rate fr and the optimal frame rate 212 is not within a prescribed range at or after a time when a switching minimum time has elapsed since the image-capturing parameters 213 was adjusted in step S1111 shown as an example in FIG. 11, the control unit 202 may return the process to step S1107 shown as an example in FIG. 11. The image-capturing control unit 1002 can hence prevent frequently changing the exposure time when the difference between the actual frame rate fr and the optimal frame rate 212 is not within a prescribed range.

### Variation Example 4 of Embodiment 3

As Variation Example 4 of the measuring instrument 100 in accordance with the present embodiment, the flickering-related information 211 may be stored in the memory unit 201.

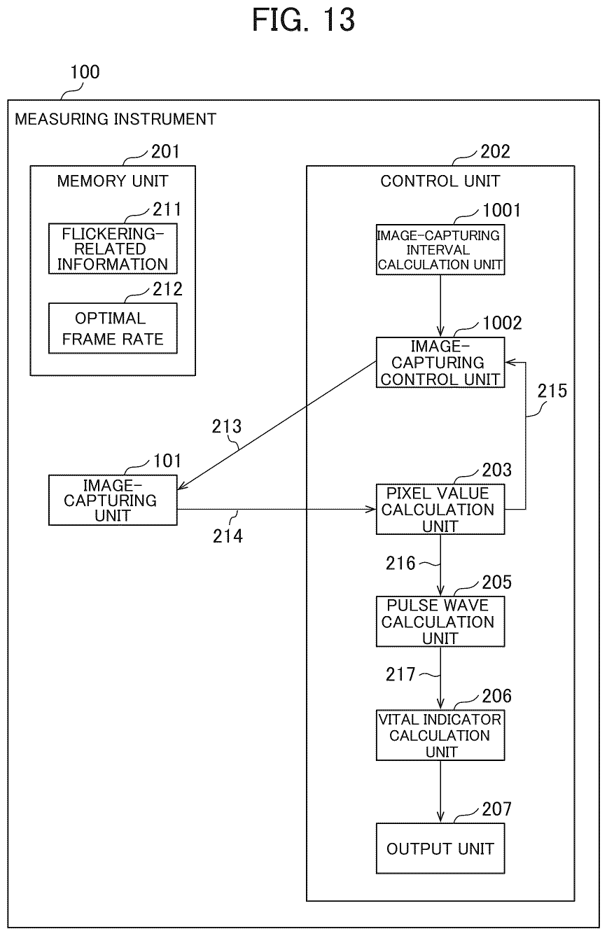

FIG. 13 is a block diagram of an exemplary structure of the measuring instrument 100 in accordance with the present variation example. The measuring instrument 100 shown as an example in FIG. 13 differs from the measuring instrument 100 shown as an example in FIG. 10 in that the measuring instrument 100 shown as an example in FIG. 13 includes no flickering detection unit 701.

The image-capturing control unit 1002 in accordance with the present variation example, determines the optimal frame rate 212 on the basis of the presence/absence of flickering represented by the flickering-related information 211 or the flickering frequency fq represented by the flickering-related information 211. The measuring instrument 100 in accordance with the present variation example can hence render the actual frame rate fr approach an optimal frame rate that is based on the flickering frequency fq specified, in accordance with illumination.

The present disclosure is not limited to the description of the embodiments above and may be altered within the scope of the claims. Embodiments based on a proper combination of technical means disclosed in different embodiments are encompassed in the technical scope of the present disclosure. Furthermore, new technological features can be created by combining different technical means disclosed in the embodiments.

What is claimed is:

1. A measuring instrument comprising:
an image-capturing unit configured to acquire a moving image by performing image capturing on a living organism;
a pixel value calculation unit configured to calculate, from each of images constituting the moving image, a representative value of pixel values of pixels in a target region containing an optical image of the living organism; and
an image-capturing control unit configured to specify an exposure time and a sensitivity based on an optimal frame rate and the representative value so that the representative value falls within a prescribed range, wherein
the image-capturing unit performs the image capturing on the living organism with the optimal frame rate, the exposure time, and the sensitivity.

2. The measuring instrument according to claim 1, wherein the image-capturing control unit adjusts the sensitivity when the representative value is below the prescribed range and the exposure time is greater than or equal to an image-capturing time interval determined from the optimal frame rate and adjusts the exposure time when the representative value is below the prescribed range and the exposure time is shorter than the image-capturing time interval.

3. The measuring instrument according to claim 1, wherein the image-capturing control unit adjusts the exposure time when the representative value is above the prescribed range and the sensitivity is lower than or equal to a lower limit level of sensitivity and adjusts the sensitivity when the representative value is above the prescribed range and the sensitivity is higher than the lower limit level of sensitivity.

4. The measuring instrument according to claim 2, wherein the adjusting of the exposure time is to specify the exposure time by selecting any of a plurality of candidate exposure times calculated from the optimal frame rate.

5. The measuring instrument according to claim 1, wherein the image-capturing control unit specifies the exposure time to a value that is less than or equal to an image-capturing time interval determined from the optimal frame rate.

6. The measuring instrument according to claim 2, wherein when the representative value is below the prescribed range, the exposure time is greater than or equal to the image-capturing time interval, and the sensitivity is lower than an upper limit level of sensitivity, the adjusting of the sensitivity involves specifying a new sensitivity that is higher than the sensitivity.

7. The measuring instrument according to claim 3, wherein when the representative value is above the prescribed range and the sensitivity is higher than the lower limit level of sensitivity, the adjusting of the sensitivity involves specifying a new sensitivity that is lower than the sensitivity.

8. A measuring method comprising:
a step of acquiring a moving image by performing image capturing on the living organism;
a step of calculating, from each of images constituting the moving image, a representative value of pixel values of pixels in a target region containing an optical image of the living organism; and
a step of specifying an exposure time and a sensitivity based on an optimal frame rate and the representative value so that the representative value falls within a prescribed range, wherein
in the step of acquiring the moving image, the image capturing is performed on the living organism with the optimal frame rate, the exposure time, and the sensitivity.

9. A computer-readable storage medium containing a program configured to cause a computer to perform:
a function of acquiring a moving image by performing image capturing on a living organism;
a function of calculating, from each of images constituting the moving image, a representative value of pixel values of pixels in a target region containing an optical image of the living organism;
a function of specifying an exposure time and a sensitivity based on an optimal frame rate and the representative value so that the representative value falls within a prescribed range; and
a function of calculating a pulse wave signal from a temporal change in the representative value, wherein
in the function of acquiring the moving image, the image capturing is performed on the living organism with the optimal frame rate, the exposure time, and the sensitivity.

10. The measuring instrument according to claim 3, wherein the adjusting of the exposure time is to specify the exposure time by selecting any of a plurality of candidate exposure times calculated from the optimal frame rate.
